(19) United States
(12) Patent Application Publication
(10) Pub. No.: US 2008/0260670 A1
(43) Pub. Date: Oct. 23, 2008

(54) Organic Compounds

(75) Inventors: Andreas Natsch, Uetikon (CH); Samuel Derrer, Fallanden (CH)

Correspondence Address: NORRIS, MCLAUGHLIN & MARCUS, 875 THIRD AVE, 18TH FLOOR, NEW YORK, NY, 10022, US

(73) Assignee: GIVAUDAN SA, Vernier (CH)

(21) Appl. No.: 12/066,871

(22) PCT Filed: Sep. 15, 2006
(86) PCT No.: PCT/CH06/00502

§ 371 Date: Mar. 14, 2008

(30) Foreign Application Priority Data
Sep. 21, 2005 (GB) 0519171.3

(51) Int. Cl. A61K 8/18; C07C 69/52; A61Q 13/00; C11D 3/20; A23L 1/22; A61Q 15/00

(52) U.S. Cl. 424/65; 560/205; 560/129; 512/26; 426/534; 510/276; 424/69

(57) ABSTRACT

A flavor or fragrance composition containing 3-hydroxy-4-methylalkanoic acids, 4-methylalk-3-enoic acids and/or their esters of formula (I)

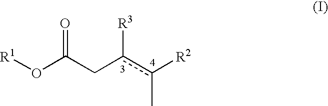

wherein R¹, R² and R³ have the same meaning as given in the description is disclosed.

The present invention refers to 3-hydroxy-4-methylalkanoic acids, 4-methylalk-3-enoic acids and their esters, and to flavor and fragrance compositions containing one or more of these compounds.

In the flavor and fragrance industry there is an ongoing demand for naturally occurring ingredients which are suitable for the creation of flavor and fragrance compositions.

Within the scope of extensive research on human body odor a new class of methyl-alkanoic/alkenoic acids have been discovered which are released by N_{α}-acyl-glutamine-aminoacylase from odorless axilla secretions. Surprisingly it has been found that these compounds possess fruity, woody, agrestic, and pungent-like odor notes, suitable for use in flavor and fragrance compositions.

Accordingly, the present invention provides in one of its aspects a flavour or fragrance composition comprising a compound of formula (I)

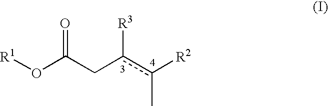

wherein

R¹ is hydrogen or C₁-C₃ alkyl, e.g. methyl, ethyl or isopropyl;

R² is linear or branched C₂-C₅ alkyl, e.g. ethyl, n-propyl, n-butyl, and n-pentyl; and
I) R³ is hydrogen and the bond between C-3 and C-4 together with the dotted line represents a double bond wherein the double bond is of E or Z configuration; or
II) R³ is hydroxyl and the bond between C-3 and C-4 is a single bond.

A particularly preferred composition according to the invention may comprise a compound of formula (I) selected from 3-hydroxy-4-methylhexanoic acid, 3-hydroxy-4-methylheptanoic acid, 3-hydroxy-4-methyloctanoic acid, isopropyl 3-hydroxy-4-methylhexanoate, methyl 3-hydroxy-4-methyloctanoate, ethyl 3-hydroxy-4-methylheptanoate, (E)-4-methyl-oct-3-enoic acid, (Z)-4-methyl-oct-3-enoic acid, (E)-4-methyl-non-3-enoic acid, (Z)-4-methyl-non-3-enoic acid, ethyl 4-methyl-oct-3-enoate, and methyl 4-methyl-non-3-enoate.

Compounds of formula (I) may be used in combination with other known flavorant or odorant molecules selected from the extensive range of natural and synthetic molecules currently available and/or in admixture with one or more ingredients or excipients conventionally used in conjugation with odorants or flavorants in fragrance or flavor compositions, for example carrier materials, and other auxiliary agents commonly used in the art. Such ingredients are, for example, described in “Perfume and Flavor Materials of Natural Origin”, S. Arctander, Ed., Elizabeth, N.J., 1960; “Perfume and Flavor Chemicals”, S. Arctander, Ed., Vol. I & II, Allured Publishing Corporation, Carol Stream, USA, 1994.

In one embodiment, the compounds of formula (I) may be used in fragrance applications, e.g. in any field of fine and functional perfumery, such as perfumes, household products, laundry products, body care products, e.g. deodorants, and cosmetics. These compounds are particularly well suited for the creation of masculine perfumes.

In another embodiment, the compounds of formula (I) may be used in flavor applications and are useful in modifying for example, dairy flavors such as butter or cheese flavors, milk, beef, chicken, meat, seasonings and exotic fruit flavors, e.g. papaya, passionfruit, mango and Durian.

In flavorant applications, the compounds of the formula (I) may be present in compositions in amounts ranging from 1 ppm up to 30 weight %, more preferably from 1.5 ppm to about 25 weight % based on the flavor composition and about 0.1 ppb to about 1 weight %, preferably about 1 ppb to about 0.5 weight % based on the finished product, such as margarine, cooking fats, oils, bakery or soaps, yogurts, candies, chewing gum or pharmaceutical products.

When used in fragrance applications, compounds of the formula (I) may be employed in amounts, for example, from about 0.0001 to about 1 weight percent. The preferred concentrations in the field of fine fragrance vary between about 0.001 and 0.01 weight percent. However, the values should not be limiting on the present invention, since the experienced perfumer may also achieve effects with even lower concentrations or may create novel accords with even higher amounts.

In a further aspect the compounds of formula (I) wherein R¹ is hydrogen and R² is a linear C₂-C₅ alkyl residue may be used for the reconstitution of axilla odor for the evaluation of deodorizing effects of odor masking compositions, such as deodorants.

Because of the newly discovered acids it is now possible to accurately reproduce the human axilla odor together with the compounds which have already been described in the literature by Natsch et. A I, Chemistry and Biodiversity, 2004, 1(7), 1058-1072.

Accordingly, the present invention refers in a further aspect to a reconstitution of cleaved human body axilla secretion comprising
a) 3-hydroxy-3-methyl hexanoic acid;
b) 3-methyl-hex-2(E)-enoic acid;
c) at least one compound of formula (I)

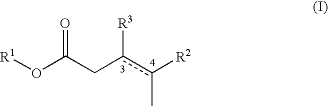

wherein R¹ is hydrogen;
R³ is hydroxyl and the bond between C-3 and C-4 is a single bond; and
R² is ethyl, n-propyl, n-butyl, or n-pentyl;
or
R³ is hydrogen and the bond between C-3 and C-4 together with the dotted line represents a double bond wherein the double bond is of the E or Z configuration;
and R² is n-butyl or n-pentyl;
d) optionally at least one sulfur compound selected from the list of 3-sulfanylhexan-1-ol, 2-methyl-3-sulfanylbutan-1-ol, 3-sulfanyl-pentan-1-ol, and 3-methyl-3-sulfanyl-hexan-1-ol; and
e) optionally at least one steroidal compound selected from the list of 5α-androst-16-en-3-one and 5α-androst-16-en-3α-ol.

In a certain embodiment the reconstitution comprises about 70 to about 85 weight % of a) plus b); about 5 to 15 weight % of at least one compound of formula (I) as defined under c); up to 1 weight %, e.g. 0.1-1 weight %, of at least one compound as defined under d); and up to I weight %, e.g. 0.1-1 weight %, of at least one compound as defined under e). For the evaluation of deodorizing effects a diluted composition comprising about 1 to 0.1 weight % of the reconstitution may be used. For the dilution solvents such as dipropyleneglycol or ethanol are suitable.

In an other embodiment the present invention refers to a method of reconstituting axilla odor for the purpose of evaluation the deodorizing effect of an odor masking composition comprising the step of admixing the reconstitution of cleaved human body axilla secretion as defined hereinabove with the odor masking composition and then evaluating either immediately or after a certain time, e.g. after 4 to 6 hours, the odor of the mixture.

The term “reconstitution”, as used herein, refers to the reproduction of a human axilla odor which is very close to the odor naturally occurring but is not necessarily exactly the same as in nature.

The compounds of formula (I) may be synthesized from commonly available starting materials and reagents according to synthetic protocols known in the art.

There now follows a series of non-limiting examples that illustrate the invention.

### EXAMPLE 1

### 3-Hydroxy-4-methylhexanoic acid

Under an atmosphere of nitrogen, a stirred solution of diisopropylamine (10.1 g, 0.1 mol) in tetrahydrofuran (50 ml) was cooled to 0° C. and n-butyllithium solution (2.7 N in heptane, 27 ml, 0.1 mol) was added dropwise within 10 minutes. The resulting solution was allowed to warm to room temperature and stirring continued at this temperature for approximately 30 minutes. The so obtained lithium diisopropylamide solution was then cooled to −10° C. and a solution of acetic acid (3.0 g, 0.05 mol) in tetrahydrofuran (15 ml) was added dropwise and under vigorous stirring within 15 minutes. Subsequently, the mixture was slowly warmed to 35° C. and kept at this temperature for 20 minutes. The resulting suspension was re-cooled to −20° C. and a solution of 2-methyl-butyraldehyde (0.05 mol, 4.3 g) was added within 10 minutes at the same temperature. The mixture was subsequently allowed to warm to room temperature and continued to stir for about 1 hour before quenching the reaction by pouring it into ice/water (250 ml). The neutral parts were then extracted with MTBE (2×100 ml). The aqueous phase was acidified with sulfuric acid (2 N, 70 ml) and extracted with MTBE (3×100 ml). The organic phases were washed with water (2×50 ml) and brine (50 ml), dried over sodium sulfate and concentrated in vacuo to obtain the crude 3-hydroxy-4-methylhexanoic acid (2.8 g, 38%, mixture of two diastereoisomers).

IR (film): ˜3400m (br, OH), ˜3500-2500m (br, CO—OH), 2963s, 2934m, 2878m, 1706vs (CO), 1462w, 1405m, 1278m, 1180s, 1051m, 1012m.

¹H-NMR (400 MHz, CDCl₃): 8.1-7.3 (2H, br s, OH and CO—OH); 4.02-3.88 (1H, m, CH(3)); 2.55-2.41 (2H, CH₂(2)); 1.61-1.42 (2H, m, CH(4) and CHH(5); 1.24-1.12 (1H, m, CHH(5); 0.96-0.86 (6H, m, Me-C(4) and CH₃(6)).

¹³C-NMR (100 MHz, CDCl₃): major diastereoisomer: 178.0 (s, CO); 71.2 (d, C(3)); 39.7 (d, C(4)); 38.7 (t, C(2)); 25.3 (t, C(5)); 13.7 (q, Me-C(4)); 11.6 (q, C(7)); minor diastereoisomer: 178.1 (s, CO); 71.7 (d, C(3)); 39.7 (q, C(4)); 37.7 (t, C(2)); 24.9 (t, C(5)); 14.3 (q, Me-C(4)); 11.4 (q, C(7)). MS (EI): 128 (2, [M−H₂O]⁺), 110 (4, [M-2H₂O]⁺), 89 (100), 71 (77), 57 (29), 43 (46), 29 (28).

Odour description: technical, ethereal, acid, pungent, slightly fruity.

### EXAMPLE 2

### 3-Hydroxy-4-methylheptanoic acid

Obtained according to the general procedure as described in Example 1 from acetic acid and 2-methyl-pentanal (5.0 g) as a yellow oil (4.0 g, 50%, mixture of two diastereoisomers).

IR (film): ˜3500-2500m (br, OH and CO—OH), 2959s, 2931m, 2874m, 1708vs (CO), 1459w, 1407m, 1288m, 1182s, 1035m.

¹H-NMR (400 MHz, CDCl₃): 8.0-7.4 (2H, br s, OH and CO—OH); 4.01-3.89 (1H, m, CH(3)); 2.56-2.41 (2H, CH₂(2)); 1.72-1.04 (5H, several m, CH(4), CH₂(5) and CH₂(6)); 0.95-0.82 (6H, m, Me-C(4) and CH₃(7)).

¹³C-NMR (100 MHz, CDCl₃): major diastereoisomer: 177.6 (s, CO); 71.5 (d, C(3)); 38.6 (t, C(2)); 37.6 (d, C(4)); 34.8 (t, C(5)); 20.2 (t, C(6)); 14.2 (q, Me-C(4)); 14.1 (q, C(7)); minor diastereoisomer: 177.7 (s, CO); 72.0 (d, C(3)); 37.7 (q, C(4)); 37.6 (t, C(2)); 34.4 (t, C(5)); 20.1 (t, C(6)); 14.6 (q, Me-C(4)); 14.0 (q, C(7)). MS (EI): 161 (2, [M+H]⁺), 124 (5, [M-2H₂O]⁺), 89 (100), 71 (57), 43 (56).

Odour description: fruity, juicy, sweaty, slightly acidic.

### EXAMPLE 3

### 3-Hydroxy-4-methyloctanoic acid

Obtained according to the general procedure as described in Example I from acetic acid and 2-methyl-hexanal (5.7 g) as a yellow oil (4.1 g, 47%, mixture of diastereoisomers).

IR (film): ˜3500-2400m (br, CO—OH), 3380m (br, OH), 2957s, 2928s, 2873m, 1708vs (CO), 1461w, 1406m, 1286m, 1182s, 1040m.

¹H-NMR (400 MHz, CDCl₃): 7.3-6.5 (2H, br s, OH and CO—OH); 4.02-3.86 (1H, m, H(3)); 2.61-2.42 (2H, CH₂(2)); 1.77-1.08 (7H, several m, CH(4), CH₂(5), CH₂(6) and CH₂(7); 0.96-0.83 (6H, m, Me-C(4) and CH₃(6)).

¹³C-NMR (100 MHz, CDCl₃): major diastereoisomer: 178.1 (s, CO); 71.4 (d, C(3)); 38.7 (t, C(2)); 38.0 (d, C(4)); 32.3 (t, C(5)); 29.4 (t, C(6)); 22.9 (t, C(7)); 14.8 (q, Me-C(4)); 14.0 (q, C(8)); minor diastereoisomer: 178.2 (s, CO); 71.9 (d, C(3)); 38.1 (d, C(4)); 37.7 (t, C(2)); 31.9 (t, C(5)); 29.3 (t, C(6)); 22.9 (t, C(7)); 14.4 (q, Me-C(4)); 14.2 (q, C(8)). MS (EI): 156 (1, [M−H₂O]⁺), 114 (7, [M−HOAc]⁺), 89 (100), 71 (44), 54 (23), 43 (68), 29 (14).

Odour description: fruity, peach-apricot, sweaty, cuminic, slightly animalic.

### EXAMPLE 4

### Isopropyl 3-hydroxy-4-methylhexanoate

Obtained following the general procedure of Example 1, but employing only 1 equivalent of lithium diisopropylamide and omitting the acid-base separation/acidification work-up. Thus obtained from isopropyl acetate (10.2 g) and 2-methyl-butyraldehyde (8.6 g) was a colourless oil (9.7 g, 52%) after Kugelrohr-distillation.

IR (film): ˜3470m (br, OH), 2965m, 2936m, 2878m, 1713s (CO), 1465w, 1405m, 1374m, 1277m, 1178s, 1105vs, 1058m, 958m. ¹H-NMR (400 MHz, CDCl₃): 5.06 (1H, hept, CH(CH₃)₂)); 3.97-3.74 (1H, m, CH(3)); 3.11 and 2.96 (1H, d, J=4.3, OH); 2.48-2.33 (2H, m, CH₂(2)); 1.59-1.13 (3H, several m, CH(4) and CH₂(5)); 1.25 (6H, d, J=6.6, CH(CH₃)₂); 0.98-0.88 (6H, m, Me-C(4) and CH₃(6)). ¹³C-NMR (100 MHz, CDCl₃): major diastereoisomer: 173.0 (s, CO); 70.9 (d, C(3)); 68.0 (d C(1′)); 39.7 (d, C(4)); 39.1 (t, C(2)); 25.4 (t, C(5)); 21.7 (q, Me₂-C(1′)); 13.7 (q, Me-C(4)); 11.7 (q, C(6)). minor diastereoisomer: 173.0 (s, CO); 71.5 (d, C(3)); 68.0 (d C(1′)); 39.7 (d, C(4)); 38.1 (t, C(2)); 24.9 (t, C(5)); 21.7 (q, Me₂-C(1′)); 14.3 (q, Me-C(4)); 11.4 (q, C(6)). MS (EI): 189 (1, [M+H]⁺), 170 (1, [M−H₂O]⁺), 145 (4), 131 (27), 111 (16), 102 (8), 89 (100), 71 (32), 57 (28), 43 (62), 29 (21).

Odour description: Fruity, woody, sawdust, slightly pine, slightly sweaty.

### EXAMPLE 5

### Methyl 3-hydroxy-4-methyloctanoate

3-Hydroxy-4-methyloctanoic acid (1.2 g) was dissolved in methanol (15 ml) and treated with trimethylsilyl diazomethane (solution, 2M in hexane, 10 ml) over a period of 60 minutes. Then the reaction was quenched by the addition of acetic acid (4 ml) and the mixture concentrated in vacuo. The crude was purified by chromatography on silica gel, eluting with hexane/ethyl acetate (7:1) to obtain the required methyl 3-hydroxy-4-methyloctanoate as a colourless oil (0.8 g, 62%, mixture of diastereoisomers).

IR (film): 3475m (br, OH), 2956s, 2928s, 2873m, 1724vs (CO), 1438s, 1379m, 1278s, 1168vs, 1048m, 989s. ¹H-NMR (400 MHz, CDCl₃): 4.09-3.83 (1H, m, CH(3)); 3.71 (3H, s, O-Me)); 3.03 and 2.89 (1H, 2s, OH)); 2.49-2.38 (2H, CH₂(2)); 1.65-1.07 (7H, several m, CH(4), CH₂(5), CH₂(6) and CH₂(7); 0.93-0.84 (6H, m, Me-C(4) and CH₃(6)). ¹³C-NMR (100 MHz, CDCl₃): major diastereoisomer: 173.8 (s, CO); 71.2 (d, C(3)); 51.7 (q, O-Me)); 38.6 (t, C(2)); 38.0 (d, C(4)); 32.4 (t, C(5)); 29.3 (t, C(6)); 22.9 (t, C(7)); 14.8 (q, Me-C(4)); 14.0 (q, C(8)); minor diastereoisomer: 178.2 (s, CO); 71.7 (d, C(3)); 51.7 (q, O-Me)); 38.1 (d, C(4)); 37.6 (t, C(2)); 31.9 (t, C(5)); 29.4 (t, C(6)); 22.9 (t, C(7)); 14.2 (q, Me-C(4)); 14.0 (q, C(8)). MS (EI): 170 (1, [M−H₂O]⁺), 141 (4), 128 (7), 103 (100), 85 (6), 71 (38), 61 (25), 43 (68).

Odour description: sweaty, cuminic.

### EXAMPLE 6

### Ethyl 3-hydroxy-4-methylhentanoate

Obtained following the general procedure of Example 1, but employing only 1 equivalent of lithium diisopropylamide and omitting the acid-base separation/acidification work-up. Thus obtained from ethyl acetate (17.6 g) and 2-methyl-valeraldehyde (20.3 g) was a colourless oil (9.0 g, 24%) after Kugelrohr-distillation.

IR (film): 3466m (br, OH), 2959s, 2931m, 2874m, 1717vs (CO), 1466w, 1372m, 1285s, 1177vs, 1024vs, 984m. ¹H-NMR (400 MHz, CDCl₃): 4.17 (2H, q, J=7.2, OCH₂CH₃)); 3.91-3.88 (1H, m, CH(3)); 3.04 and 2.89 (1H, 2s, OH)); 2.51-2.37 (2H, m, CH₂(2)); 1.65-1.04 (5H, several m, —CH(4), CH₂(5) and CH₂(6)); 1.28 (3H, t, J=7.2, OCH₂CH₃)); 0.92-0.85 (6H, m, Me-C(4) and CH₃(7)). ¹³C-NMR (100 MHz, CDCl₃): major diastereoisomer: 173.5 (s, CO); 71.2 (d, C(3)); 60.6 (t, OC(1′)); 38.8 (t, C(2)); 37.7 (d, C(4)); 34.9 (t, C(5)); 20.3 (t, C(6)); 14.8 (q, C(2″)); 14.2 (q, A4e-C(4)); 14.1 (q, C(7)); minor diastereoisomer: 173.5 (s, CO); 71.7 (d, C(3)); 60.6 (t, OC(1′)); 37.8 (t, C(2)); 37.7 (d, C(4)); 34.5 (t, C(5)); 20.2 (t, C(6)); 14.8 (q, C(2″)); 14.2 (q, Me-C(4)); 14.1 (q, C(7)). MS (EI): 170 (5, [M−H₂O]⁺), 125 (19), 117 (100), 99 (16), 89 (44), 71 (75), 55 (27), 43 (46), 29 (26).

Odour description: agrestic, woody-pine, slightly cypress.

### EXAMPLE 7

### (E/Z)-4-Methyl-oct-3-enoic acid

Under an atmosphere of nitrogen, a flask was charged with 2-hexanone (25 g, 0.25 mol), (2-carboxyethyl)triphenylphosphonium chloride (100 g, 0.27 mol), tetrahydrofuran (250 ml), and 1-methyl-2-pyrrolidinone (250 ml) and the mixture cooled to 0° C. Under stirring, sodium hydride (20 g, 0.5 mol) was added in small portions over a period of 30 minutes, during which the temperature was allowed to rise to 25° C. and hydrogen evolution was observed. Gentle heating to 30-35° C. was applied until the slightly exothermic reaction started and no further heating was needed to keep the temperature above 30° C. during about 1 hour. Stirring was continued for 5 hours at room temperature, after which the hydrogen evolution ceased and the brownish mixture became more fluid again. This mixture was then poured into ice/water (1 kg) and extracted with MTBE/hexane (1:1, 3×400 ml). The aqueous phase was acidified to pH 2 with diluted sulfuric acid (˜300 ml) and extracted with hexane (2×300 ml). The combined organic phases were washed with hot water (2×200 ml) and dried over sodium sulfate (standing over night, during which the majority of remaining triphenylphosphine oxide precipitated and was filtered off). The solvent was removed under reduced pressure and further dried at 0.1 mbar for 5 h to give the desired crude mixture of (E)-4-methyl-oct-3-enoic acid and (Z)-4-methyl-oct-3-enoic acid [19.2 g, 50%, E:Z-ratio=2:1 (GLC-analysis), containing residual triphenylphosphine oxide] as a pale yellow oil.

IR (film): ˜3400-2400m (br, CO—OH), 2958m, 2929s, 2860m, 1705vs (CO), 1412m, 1379w, 1294m, 1218m, 1129w.

¹H-NMR (400 MHz, CDCl₃): 11.2 (1H, br s, OH); 5.30 (1H, tt, J=7.2, 1.4, ═CH); 3.09 (1H, dd, J=7.2, 0.8, CH₂(2)); 2.02 (2H, t, J=7.2, CH₂(5)); 1.74 (0.33.3H, s, ═CHMe, Z-isomer); 1.63 (0.66.3H, s, ═CHMe, E-isomer); 1.42-1.23 (4H, several m, CH₂(6) and CH₂(7)); 0.91 (0.33.3H, t, J=7.2, CH₃(8, Z-isomer)); 0.89 (0.66.3H, t, J=7.2, CH₃(8, E-isomer)).

¹³C-NMR (100 MHz, CDCl₃): Z-isomer: 179.2 (s, CO); 140.3 (s, C(4)); 115.2 (s, C(3)); 33.3 (t, C(2)); 31.7 (t, C(5)); 29.9 (t, C(6)); 23.3 (q, C(4)-Me); 22.6 (t, C(7)); 13.9 (q, C(8)); E-isomer: 179.2 (s, CO); 140.1 (s, C(4)); 114.6 (s, C(3)); 39.2 (t, C(5)); 33.5 (t, C(2)); 29.9 (t, C(6)); 22.3 (t, C(7)); 16.2 (q, C(4)-Me); 13.9 (q, C(8)). MS (EI): E-isomer: 156 (18, M⁺), 114 (34), 96 (61), 81 (53), 69 (94), 55 (100), 43 (44), 41 (81). GC/MS (EI): Z-isomer: 156 (20, M⁺), 114 (47), 96 (66), 81 (58), 69 (100), 55 (98), 43 (44), 41 (80).

Odor description of the isomeric mixture: pungent, green, acid, sweat-mean like.

### EXAMPLE 8

### (E/Z)-4-Methyl-non-3-enoic acid

Obtained following the general procedure as described in Example 7 from (2-carboxyethyl)triphenylphosphonium chloride and 2-heptanone (34.4 g) as a yellow oil (1.1 g, 2%, mixture of E:Z-isomers), after chromatography on silica gel.

IR (film): ˜3400-2400m (br, CO—OH), 2957m, 2927s, 2859m, 1706vs (CO), 1412m, 1379w, 1294m, 1220m, 1157w938m. ¹H-NMR (400 MHz, CDCl₃): 11.4 (1H, brs, OH); 5.31 (1H, tt, J=8.6, 1.5, ═CH); 3.09 (1H, dd, J=7.3, 1.0, CH₂(2)); 2.02 (2H, t, J=8.6, CH₂(5)); 1.74 (0.45.3H, s, ═CHMe, Z-isomer); 1.63 (0.55-3H, s, ═CHMe, E-isomer); 1.45-1.18 (6H, several m, CH₂(6), CH₂(7) and CH₂(8)); 0.88 (3H, t, J=7.1, CH₃(8)). ¹³C-NMR (100 MHz, CDCl₃): Z-isomer: 179.1 (s, CO); 140.3 (s, C(4)); 115.2 (d, C(3)); 33.3 (t, C(2)); 31.9 (t, C(7); 31.7 (t, C(5)); 27.4 (t, C(6)); 23.3 (q, C(4)-Me); 22.5 (t, C(8)); 14.0 (q, C(9)); E-isomer: 179.2 (s, CO); 140.1 (s, C(4)); 114.6 (s, C(3)); 39.5 (t, C(5)); 33.5 (t, C(2)); 31.5 (t, C(7)); 27.4 (t, C(6)); 22.5 (t, C(8)); 16.2 (q, C(4)-Me); 14.0 (q, C(9)). MS (EI): E-isomer: 170 (15, M⁺), 152 (4), 127 (9), 114 (47), 96 (35), 81 (38), 69 (100), 55 (89), 41 (76). GC-MS (EI): Z-isomer: 170 (24, M⁺), 152 (6), 127 (8), 114 (45), 96 (36), 81 (39), 69 (100), 55 (93), 41 (78).

Odour description: oily, fishy, costus-like, slightly woody, goat-like.

### EXAMPLE 9

### Ethyl 4-methyl-oct-3-enoate

A mixture of 4-methyl-3-octenoic acid (2.3 g), dicyclohexyl carbodiimide (3.4 g), 4-dimethylaminopyridine (90 mg) and ethanol (1 ml) in dichloromethane (100 ml) was stirred at room temperature for 20 h. The precipitation was filtered off and the filtrate concentrated in vacuo. The crude was purified by chromatography on silica gel, eluting with hexane/ethyl acetate (100:1) to afford ethyl 4-methyl-oct-3-enoate as a pale yellow oil (1.5 g, 54%).

IR (film): 2958m, 2930m, 2860w, 1736vs (CO), 1447w, 1367w, 1313m, 1255m, 1154s, 1029s. ¹H-NMR (400 MHz, CDCl₃): 5.32 (1H, tt, J=7.1, 1.5, ═CH); 4.14 (3H, q, J=7.3, OCH₂-Me)); 3.03 (1H, d, J=8.1, CH₂(2)); 2.02 (2H, t, J=9.0, CH₂(5)); 1.73 (0.4.3H, s, ═CHMe, Z-isomer); 1.62 (0.6-3H, s, ═CHMe, E-isomer); 1.41-1.24 (4H, several m, CH₂(6) and CH₂(7)); 1.26 (3H, t, J=7.3, OCH₂-Me)); 0.91-0.73 (m, CH₃(8)). ¹³C-NMR (100 MHz, CDCl₃): Z-isomer: 172.5 (s, CO); 139.5 (s, C(4)); 116.1 (s, C(3)); 60.4 (t, OCH₂-Me)); 33.6 (t, C(2)); 31.7 (t, C(5)); 29.9 (t, C(6)); 23.4 (q, C(4)-Me); 22.6 (t, C(7)); 14.2 (t, OCH₂-Me)); 13.9 (q, C(8)); E-isomer: 172.5 (s, CO); 139.3 (s, C(4)); 115.5 (s, C(3)); 60.4 (t, OCH₂-Me)); 39.2 (t, C(2)); 33.8 (t, C(5)); 30.0 (t, C(6)); 22.2 (t, C(7)); 16.2 (q, C(4)-Me); 14.2 (t, OCH₂-Me)); 13.9 (q, C(8));). MS (EI): E-isomer: 184, (20, M⁺), 155 (3), 142 (18), 111 (23), 96 (57), 81 (28), 69 (100), 55 (96), 41 (42), 29 (37). GC-MS (EI): Z-isomer: 184 (17, M⁺), 155 (5), 142 (21), 110 (29), 96 (63), 81 (35), 69 (92), 55 (100), 41 (42), 29 (36).

Odor description: etheral, fruity, floral.

### EXAMPLE 10

### Methyl 4-methyl-non-3-enoate

4-Methyl-non-3-enoic acid (0.7 g) was dissolved in methanol (5 ml) and treated with trimethylsilyl diazomethane (solution, 2M in hexane, 4 ml). Then the reaction was quenched by the addition of acetic acid (several drops) and the mixture concentrated in vacuo. The crude was purified by chromatographie on silica gel, eluting with hexane/ethyl acetate (50:1) to obtain the required methyl 4-methyl-non-3-enoate as a colourless oil (0.5 g, 72%, mixture of diastereoisomers).

IR (film): 2955m, 2982s, 2858w, 1740vs (CO), 1435m, 1378w, 1318w, 1257m, 1194m, 1157s, 1018m. ¹H-NMR (400 MHz, CDCl₃): 5.32 (1H, tt, J=8.0, 1.5, ═CH); 3.68 (3H, s, O-Me)); 3.05 (1H, d, J=7.6, CH₂(2)); 2.01 (2H, t, J=7.8, CH₂(5)); 1.73 (0.46.3H, s, ═CHMe, Z-isomer); 1.62 (0.54-3H, s, ═CHMe, E-isomer); 1.46-1.20 (6H, several m, CH₂(6), CH₂(7) and CH₂(8)); 0.91-0.73 (m, CH₃(9)). ¹³C-NMR (100 MHz, CDCl₃): Z-isomer: 172.9 (s, CO); 139.7 (s, C(4)); 115.9 (s, C(3)); 51.6 (q, O-Me)); 33.3 (t, C(2)); 31.9 (t, C(5)); 31.7 (t, C(6)); 27.4 (t, C(7)); 23.4 (q, C(4)-Me); 22.6 (t, C(8)); 13.9 (q, C(9)); E-isomer: 172.9 (s, CO); 139.5 (s, C(4)); 115.3 (s, C(3)); 51.6 (q, O-Me)); 39.5 (t, C(2)); 33.5 (t, C(5)); 31.7 (t, C(6)); 27.4 (t, C(7)); 23.4 (q, C(4)-Me); 22.5 (t, C(8)); 14.0 (q, C(8));). MS (EI): E-isomer: 184, (24, M⁺), 152 (12), 128 (38), 110 (64), 96 (57), 81 (52), 69 (100), 55 (98), 41 (56), 29 (24). GC-MS (EI): Z-isomer: 184 (21, M⁺), 152 (8), 128 (34), 110 (55), 96 (48), 81 (39), 69 (100), 55 (96), 41 (47), 29 (18).

Odor description: fruity, winey, metallic, slightly cheese-like, fatty-green, slightly fruity.

### EXAMPLE 11

### Fragrance Compositions

A)

| | Parts per |
|---|---|
| | weight |
| Allyl amyl glycolate | 2 |
| Ambrofix (Givaudan) at 10% in DPG | 45 |
| Boisambrene forte (1-(ethoxymethoxy)-cyclododecane) | 15 |
| Dihydro myrcenol (2,6-dimethyloct-7-en-2-ol) | 80 |
| Galaxolide ® 50 PHT (1,3,4,6,7,8-hexahydro- | 200 |
| 4,6,6,7,8,8-hexamethyl-cyclopenta-gamma-2-benzopyran) | |
| Sandalore ® (5-(2,2,3-trimethyl-3-cyclopentenyl)- | 35 |
| 3-methylpentan-2-ol) | |
| Isobutyl quinoleine-2 (2-(2-methylpropyl)quinoline) | 2 |
| Nutmeg essential oil from Indonesia | 25 |
| Cyclohexal | 120 |
| Methyl cedryl ketone (CAS 32388-55-9) | 120 |
| Basil essential oil | 2 |
| Iso E Super ® (7-acetyl, 1,2,3,4,5,6,7,8-octahydro-1,1,6,7- | 100 |
| tetramethyl naphthalene) | |
| Musk Ketone (4′-tert-Butyl-2′,6′-dimethyl-3′,5′- | 4 |
| dinitroacetophenone) | |
| Bergamot essential oil | 45 |
| Vanillin at 10% in DPG | 5 |
| Ccyclal C (2,4-Dimethylcyclohex-3-enecarbaldehyde) | 4 |
| at 10% in DPG | |
| Mandarin essential oil | 15 |
| 3-Phenylprop-2-enal | 1 |
| Coumarine pure crist. | 2 |
| Dipropylene glycol (DPG) | 69 |

The addition of 9 parts (E/Z)-4-methyl-3-octenoic acid at 10% in DPG makes the above composition more sensual and attractive. It imparts an agrestic-animalic effect, enhances the carnal woody musky spicy accord, and keeps the lovely freshness of the fragrance.

B)

| | Parts per weight |
|---|---|
| Acetyl isoeugenol crist. | 8 |
| Adoxal (2,6,10-trimethyl-9-undecenal) at 10% in DPG | 1 |
| Aldehyde C 12 Lauric (dodecenal) at 10% in DPG | 1 |
| Peche pure (5-heptyldihydro-2(3H)-furanone) | 3 |
| Radjanol (2-ethyl-4-(2,2,3-trimethyl-3-cyclopenten-1-yl)-2-buten-1-ol) | 10 |
| Benzyl propionate | 5 |
| Dihydro myrcenol crude | 35 |
| Ethyl 2-methylbutyrate at 10% in DPG | 1 |
| Florhydral ® (3-(3-isopropylphenyl)butanal) | 3 |
| Florol ® (tetrahydro-4-methyl-2-(2-methylpropyl)-2H-pyran-4-ol) | 45 |
| N 112 (1-(4-hydroxyphenyl)-butan-3-one) | 5 |
| Nirvanolide ((10Z)-13-methyloxacyclopentadec-10-en-2-one) | 150 |
| Irone Alpha (CAS 79-69-6) at 1% in DPG | 25 |
| Jasmolactone (Firmenich) at 10% in DPG | 25 |
| Majantol (2,2-dimethyl-3-(3-methyl phenyl)-propanol) | 35 |
| Precyclemone B (1-methyl-4-(4-methyl-3-pentenyl)-3-cyclohexene- | 25 |
| 1-carbaldehyde) at 10% in DPG | |
| Sandalore ® (5-(2,2,3-trimethyl-3-cyclopentenyl)-3-methylpentan-2-ol) | 20 |
| Timberol (2,2,6-trimethyl-α-propyl-cyclohexanepropanol) | 1 |
| at 10% in DPG | |
| Undecavertol (4-methyl-3-decen-5-ol) at 10% in DPG | 8 |
| Vanillin at 1% in DPG | 15 |
| Agrumex (2-(1,1-dimethylethyl)-cyclohexyl acetate) | 20 |
| Para-tert-butylcyclohexyl acetate | 10 |
| Piconia (IFF) at 10% in DPG | 1 |
| Dipropylene glycol (DPG) | 34 |
| Cyclal C (2,4-dimethylcyclohex-3-enecarbaldehyde) at 1% in DPG | 4 |

The addition of 10 parts methyl 3-hydroxy-4-methyl-octanoate at 10% in DPG to the above composition helps to develop the sensuality of its floral-musky accord. It enriches also the fruity top note and enhances the touch of spiciness.

### EXAMPLE 12

### Flavor Applications

A) Ghee flavor

| | parts by weight |
|---|---|
| Butyric acid | 200.00 |
| Hexanoic acid | 50.00 |
| Octanoic acid | 50.00 |
| Decanoic acid | 100.00 |
| Butane-2,3-dione | 2.00 |
| 2-Undecanone | 1.00 |
| gamma Nonalactone | 10.00 |
| Skatol | 0.05 |
| Vegetable oil | qsp 1000.00 |

To the standard ghee flavor above about 30 ppm to about 8 weight % of 4-methyl-3-hydroxy-betanoic acid or about 500 ppm to about 25 weight % of 3-hydroxy-4-methyl-hexanoic acid may be added. The resulting flavor composition may be added, for example, to cooking fats or oil and margarine in a range of about 20 to 2000 ppm.

B) Butter flavor

| | parts per weight |
|---|---|
| Decanoic acid | 10 |
| Butane-2,3-dione | 50 |
| delta Decalactone | 50 |
| delta Dodecalactone | 20 |
| Vegetable oil | qsp 1000 |

To the standard butter flavor above about 10 ppm to about 1 weight % of 4-methyl-3-hydroxy-betanoic acid or about 100 ppm to about 10 weight % of 3-hydroxy-4-methyl-hexanoic acid may be added. The resulting flavor composition may be added, for example, to cooking fats or oil and margarine in a range of about 20 to 2000 ppm, or e.g. to cookies, biscuits or cakes in a range of about 200 to 5000 ppm.

C) Cheese flavor

| | Parts per weight |
|---|---|
| Acetic acid | 30.0 |
| Butyric acid | 250.0 |
| Octanoic acid | 100.0 |
| Decanoic acid | 170.0 |
| Dodecanoic acid | 30.0 |
| Tetradecanoic acid | 30.0 |
| Hexadecanoic acid | 20.0 |
| Methyl-4 octanoic acid | 10.0 |
| Ethyl decanoate | 5.0 |
| delta Decalactone | 0.5 |
| gamma Dodecalactone | 0.5 |
| Vegetable oil | qsp 1000.0 |

To the standard cheese flavor above about 20 ppm to about 5 weight % of 4-methyl-3-hydroxy-betanoic acid or about 500 ppm to about 20 weight % of 3-hydroxy-4-methyl-hexanoic acid may be added. The resulting flavor composition may be added, for example, to cooking fats or oils and margarine in a range of about 20 to 8000 ppm, or e.g. to cookies, biscuits or cakes in a range of about 200 to 10000 ppm.

1. A fragrance or flavor composition comprising a compound of formula (I) 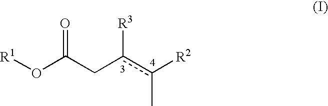
wherein
R¹ is hydrogen or C₁-C₃ alkyl;
R² is linear or branched C₂-C₅ alkyl; and
I) R³ is hydrogen and the bond between C-3 and C-4 together with the dotted line represents a double bond, wherein the double bond is in E or Z configuration; or
II) R³ is hydroxyl and the bond between C-3 and C-4 is a single bond.

2. A fragranced product comprising a compound of formula (I) as defined in claim 1 in an amount ranging from 0.0001 to 1 weight %.

3. A product according to claim 2 wherein the product is selected from the list consisting of perfume, household products, laundry products, body care products and cosmetics.

4. A product according to claim 3 wherein the body care product is a deodorant.

5. A flavorant application comprising a compound of formula (I) as defined in claim 1 in an amount ranging from 1 ppm up to 30 weight %.

6. A flavorant application according to claim 5 comprising a compound of formula (I) as defined in claim 1 in an amount ranging from 0.1 ppb up to 1 weight % based on the finished product

7. A method of improving a flavour or fragrance composition comprising the step of adding thereto one or more compounds of the formula (I) as defined in claim 1.

8. A reconstitution of cleaved human body axilla secreation comprising
a) 3-hydroxy-3-methyl hexanoic acid;
b) 3-methyl-hex-2(E)-enoic acid;
c) at least one compound of formula (I) 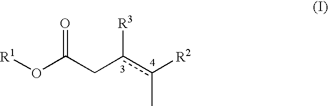
wherein R¹ is hydrogen;
R³ is hydroxyl and the bond between C-3 and C-4 is a single bond; and
R² is ethyl, n-propyl, n-butyl, or n-pentyl;
or
R³ is hydrogen and the bond between C-3 and C-4 together with the dotted line represents a double bond wherein the double bond is of the E or Z configuration; and R² is n-butyl or n-pentyl;
d) optionally at least one sulfur compound selected from the list of 3-sulfanylhexan-1-ol, 2-methyl-3-sulfanylbutan-1-ol, 3-sulfanyl-pentan-1-ol, and 3-methyl-3-sulfanyl-hexan-1-ol; and
e) optionally at least one steroidal compound selected from the list of 5α-androst-16-en-3-one and 5α-androst-16-en-3α-ol.
